(19) United States
(12) Patent Application Publication
(10) Pub. No.: US 2007/0118137 A1
(43) Pub. Date: May 24, 2007

(54) METHOD OF MANUFACTURING AN ORTHOPAEDIC REAMER

(75) Inventors: Todd A. Wolford, Goshen, IN (US); Ray W. Hathaway, Pierceton, IN (US)

Correspondence Address: TAYLOR & AUST, P.C., 142 SOUTH MAIN STREET, P. O. BOX 560, AVILLA, IN, 46710, US

(73) Assignee: Symmetry Medical, Inc.

(21) Appl. No.: 11/626,154

(22) Filed: Jan. 23, 2007

Related U.S. Application Data
(62) Division of application No. 10/662,559, filed on Sep. 15, 2003.

(51) Int. Cl. A61B 17/00

(52) U.S. Cl. 606/80

(57) ABSTRACT

A method of manufacturing an orthopaedic reamer includes the steps of: forming a shell having a cutting face; cutting a plurality of openings in the shell, each opening defining a cutting edge of a tooth and a pair of relief cuts extending transverse from opposite ends of the cutting edge, each relief cut terminating at a base end; and bending each cutting tooth in a single bending operation about an axis extending between the base ends, with each cutting edge having a shape after the bending step which is predefined by the cutting step.

### BACKGROUND OF THE INVENTION

1. Field of the Invention

The present invention relates to orthopaedic reamers, and, more particularly, to cutting teeth on orthopaedic reamers.

2. Description of the Related Art

An orthopaedic reamer typically includes a rotating body with teeth extending therefrom which cut surface of a bone. For example, an acetabular reamer includes a hemispherical shell with a plurality of radially outward extending teeth which ream an acetabulum for receiving an acetabular implant.

With an acetabular reamer as described above, a generally hemispherical shell has a radial outward shape corresponding to the shape of the acetabulum to be formed. The teeth are typically formed using multiple stamping and punching operations for each tooth. Forming each cutting tooth in multiple steps increases the cost of the reamer. Moreover, stamping the teeth results in each tooth having a domed or partially domed configuration, thereby making it necessary to overlap a next laterally adjacent tooth in order to shape the bone without significant scalloping or other undesirable cutting characteristics.

What is needed in the art is an orthopaedic reamer with teeth which are formed in a simpler manner and have a shape which more closely approximates the shape to be cut.

### SUMMARY OF THE INVENTION

The present invention provides an orthopaedic reamer with teeth which are formed in a simple and reliabe manner. Predefined openings are cut in a shell of the reamer to define cutting edges and transverse relief cuts. The cutting teeth are bent in a single bending operation about an axis at the base of the relief cuts.

The invention comprises, in one form thereof, a method of manufacturing an orthopaedic reamer, including the steps of: forming a shell having a cutting face; cutting a plurality of openings in the shell, each opening defining a cutting edge of a tooth and a pair of relief cuts extending transverse from opposite ends of the cutting edge, each relief cut terminating at a base end; and bending each cutting tooth in a single bending operation about an axis extending between the base ends, with each cutting edge having a shape after the bending step which is predefined by the cutting step.

An advantage of the present invention is that the cutting edges are cut in the reamer, prior to being bent, with a shape which translates into a desired shape after being bent.

Another advantage is that the cutting teeth are bent in a single bending operation.

Yet another advantage is that the cutting teeth may be formed with different desired shapes after being bent.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of an embodiment of an orthopaedic reamer of the present invention;

FIG. 2 is a plan view of one of the cutting teeth shown in FIG. 1;

FIG. 3 is a sectional view of the cutting tooth taken along line 3-3 in FIG. 2, prior to being bent;

FIG. 4 is a side, fragmentary view of the cutting tooth of FIG. 3, after being bent; and

FIG. 5 is a perspective view of another embodiment of an orthopaedic reamer of the present invention.

Corresponding reference characters indicate corresponding parts throughout the several views. The exemplifications set out herein illustrate one preferred embodiment of the invention, in one form, and such exemplifications are not to be construed as limiting the scope of the invention in any manner.

### DETAILED DESCRIPTION OF THE INVENTION

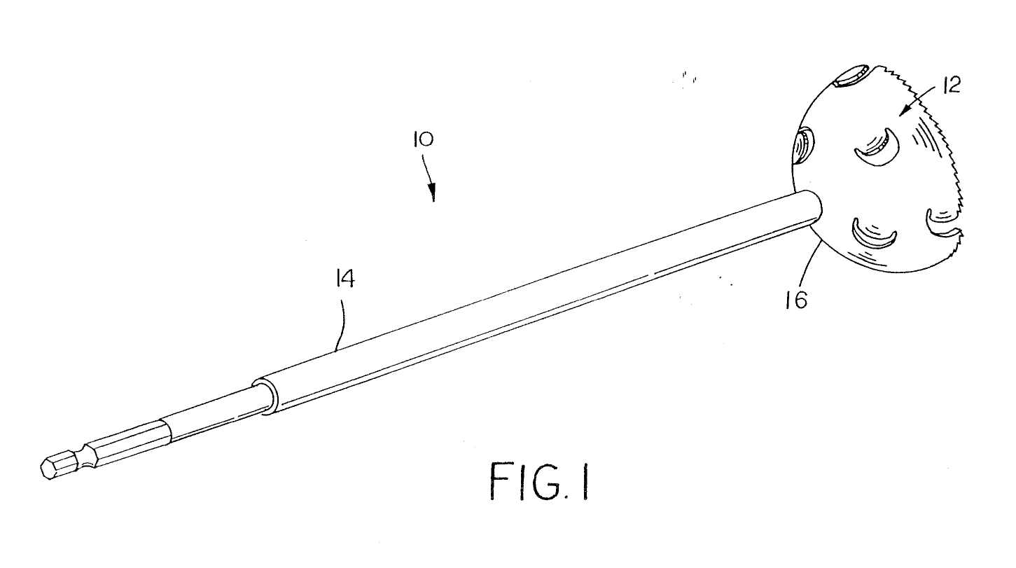

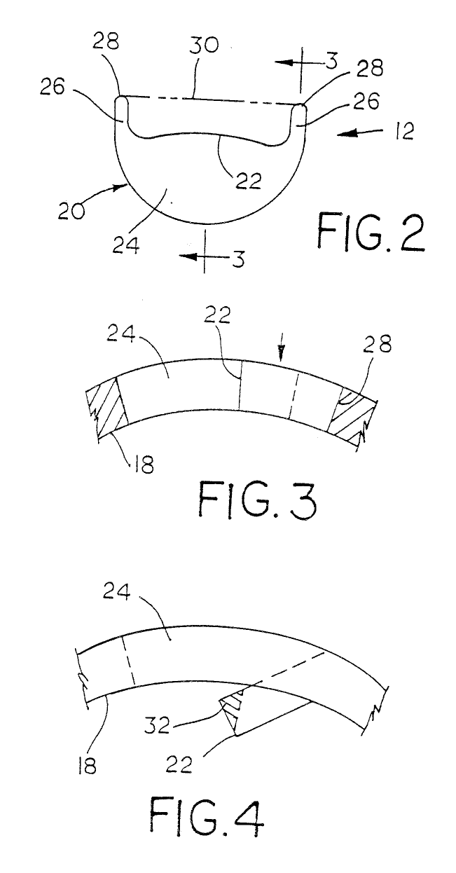

Referring now to the drawings, and more particularly to FIGS. 1-4, there is shown an embodiment of an orthopaedic reamer 10 of the present invention, which is manufactured by a method of manufacturing an orthopaedic reamer as will be described hereinafter. In the embodiment shown, orthopaedic reamer 10 is used for shaping the proximal femoral head of a patient. Configured as such, orthopaedic reamer 10 has a concave shape with a plurality of cutting teeth 12 which extend radially inward.

More particularly, orthopaedic reamer 10 includes a stem 14 attached to a hemispherical shell 16. Shell 16 has a cutting face 18 on a radially inward side which is positioned adjacent to a surface of a bone to be cut.

A plurality of openings 20 are cut at predefined locations in shell 16. For example, openings 20 may be positioned in a spiral configuration, offset configuration, etc., depending upon the application. Each opening 20 defines a cutting edge 22 of a cutting tooth 12. A clearance opening 24 is positioned adjacent the leading edge of cutting edge 22 for allowing bone chips to pass therethrough. A pair of relief cuts 26 extend transverse from opposite ends of cutting edge 22, in a direction generally away from clearance opening 24. Each relief cut 26 terminates at a base end 28, which defines an axis 30 therebetween about which cutting tooth 12 is bent in a single bending operation, as will be described in more detail hereinafter.

During manufacture of orthopaedic reamer 10, shell 16 is formed with cutting face 18 at either the radial inner surface or radial outer surface, depending upon the application. In the embodiment shown in FIGS. 1-4, cutting face 18 is formed on the radial inner surface. Openings 20 are then cut into shell 16 at selected locations. Cutting edge 22 is formed with a predefined shape which can be concave, convex, straight or a combination thereof, depending upon the desired final translated shape of cutting edge 22. With conventional manufacturing techniques, each cutting tooth is stamped in multiple stamping operations. In contrast, the manufacturing method of the present invention uses two primary design criteria to establish the final shape of each cutting tooth 12. First, the shape of each cutting edge is formed not with respect to the shape prior to bending, but rather with regard to the desired final shape after bending. Second, each cutting tooth is bent in a single bending operation about an axis extending between the base of relief cuts 26. Preferably, each opening 20 is cut with a fast and clean manufacturing process, such as by laser cutting. By laser cutting the openings and bending each tooth in a single bending operation, the plurality of cutting teeth 12 may be quickly, inexpensively and accurately formed in shell 16. A portion of each cutting edge 22 may be removed, as shown in section lines 32 in FIG. 4, to define a sharper cutting edge.

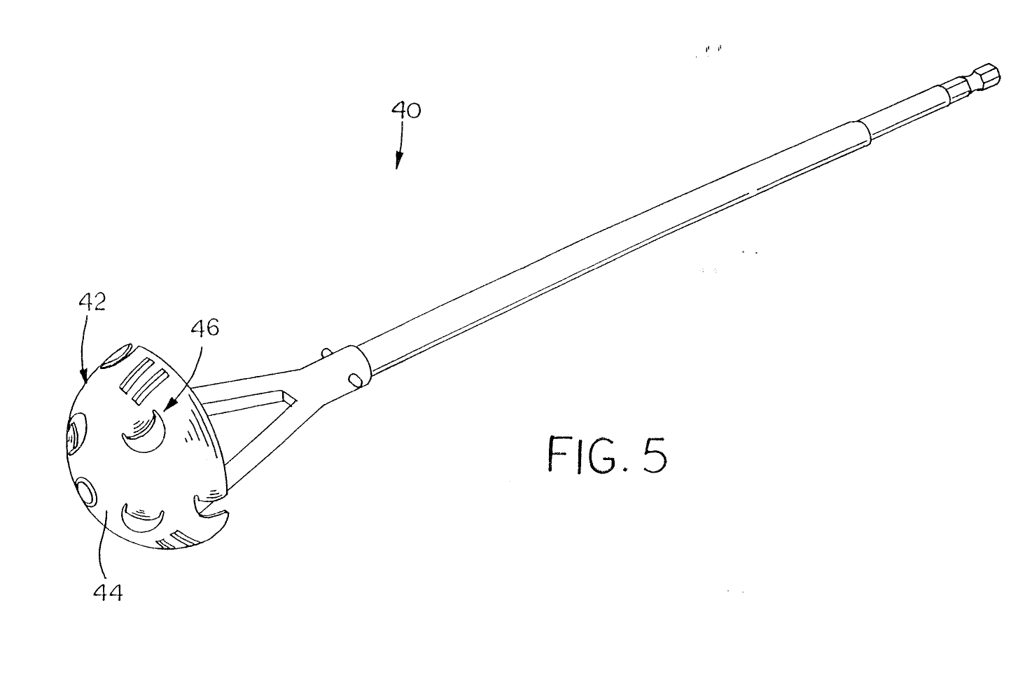

FIG. 5 illustrates another embodiment of an orthopaedic reamer 40 of the present invention. Orthopaedic reamer 40 includes a hemispherical shell 42 with a convex cutting face 44. A plurality of teeth 46 are formed in cutting face 44 of shell 42 in a manner similar to the manufacturing method described above with regard to orthopaedic reamer 10, the primary difference being that teeth 46 extend radially outward from cutting face 44.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A method of manufacturing an orthopaedic reamer, comprising the steps of:
forming a shell having a convex side and a concave side and a cutting face in said concave side;
cutting a plurality of openings in said shell, each said opening defining a cutting edge of a tooth and a pair of relief cuts extending transverse from opposite ends of said cutting edge, each said relief cut terminating at a base end; and
bending each said cutting edge in a single bending operation about an axis extending between said base ends, each said cutting edge having a shape after said bending step which is predefined by said cutting step, wherein each said cutting edge extends radially inwardly from said convex side to said concave side.

2. The method of manufacturing an orthopaedic reamer of claim 1, wherein said cutting edge has a shape after said bending step which is different than a shape of said cutting edge after said cutting step.

3. The method of manufacturing an orthopaedic reamer of claim 2, wherein each said cutting edge has a shape after said bending step which is one of curved and straight as viewed from a leading edge of said cutting tooth.

4. (canceled)

5. (canceled)

6. The method of manufacturing an orthopaedic reamer of claim 1, wherein said cutting step includes forming a clearance opening adjacent a leading edge of each said cutting edge.

7. The method of manufacturing an orthopaedic reamer of claim 6, wherein each said clearance opening is a generally hemi-circular opening.

8. A method of forming cutting teeth in an orthopaedic reamer, the orthopaedic reamer including a shell having a cutting face, said method comprising the steps of:
cutting a plurality of openings in the shell, each said opening defining a cutting edge of a tooth and a pair of relief cuts extending transverse from opposite ends of said cutting edge, each said relief cut terminating at a base end; and
bending each said cutting edge in a single bending operation about an axis extending between said base ends, each said cutting edge having a shape after said bending step which is predefined by said cutting step, wherein said shell has a convex side and a concave side and said cutting face being in said concave side and each said cutting edge extends radially inwardly from said convex side to said concave side.

9. The method of forming cutting teeth of claim 8, wherein said cutting edge has a shape after said bending step which is different than a shape of said cutting edge after said cutting step.

10. The method of forming cutting teeth of claim 9, wherein each said cutting edge has a shape after said bending step which is one of curved and straight as viewed from a leading edge of said cutting tooth.

11. (canceled)

12. (canceled)

13. The method of forming cutting teeth of claim 8, wherein said cutting step includes forming a clearance opening adjacent a leading edge of each said cutting edge.

14. The method of forming cutting teeth of claim 13, wherein each said clearance opening is a generally hemi-circular opening.

15. (canceled)

16. (canceled)

17. (canceled)

18. (canceled)
